(19) United States
(12) Patent Application Publication
(10) Pub. No.: US 2026/0240461 A1
(43) Pub. Date: Aug. 20, 2026

(54) Mechanical Lock for a Laser-Based Photoplethysmographic Sensor

(71) Applicant: Zynex Monitoring Solutions, Inc., Englewood, CO (US)

(72) Inventor: Jake E. Nickoloff, Thornton, CO (US)

(73) Assignee: Zynex Monitoring Solutions, Inc, Englewood, CO (US)

(21) Appl. No.: 19/054,383

(22) Filed: Feb. 14, 2025

(51) Int. Cl. A61B 5/1455; A61B 5/024

(52) U.S. Cl. CPC A61B 5/14552; A61B 5/02427; A61B 2562/12; A61B 2562/227

(57) ABSTRACT

A photoplethysmographic sensor for a laser-based pulse oximeter has an enclosure and a sensor connector. The sensor connector including a stationary portion and a sliding portion, the stationary portion fixed in position with respect to the enclosure, the sliding portion coupled to the stationary portion and free to slide with respect to the stationary portion, the sensor connector stable in at least an open position and an engaged position and movable between the open and engaged positions through application of an external force, and the sensor connector adapted to retain a distal connector when in the engaged position.

### FIELD OF THE INVENTION

This invention is in the field of photoplethysmography.

### BACKGROUND OF THE INVENTION

In the science of photoplethysmography, light is used to illuminate or trans-illuminate living tissue for the purpose of providing noninvasive measurements of blood analytes including but not limited to the levels of arterial oxyhemoglobin, carboxyhemoglobin, methemoglobin, reduced hemoglobin, and/or total hemoglobin. Additionally, photoplethysmography can be designed to measure various hemodynamic parameters, and/or tissue properties including, but not limited to, heart rate, respiratory rate, and perfusion.

In this monitoring modality multiple different spectral bands of light are directed, by a photoplethysmographic sensor, into living tissue (the “tissue-under-test”) and a portion of the light that is not absorbed by the tissue or scattered in some other direction is detected by a photodetector a short distance from the point at which the light entered the tissue. When light, at wavelengths that can be absorbed by hemoglobin or other components of arterial blood, passes through living tissue the light is modulated by the pulsatile arterial blood flow. The pulsatile (or photoplethysmographic) light signals exiting the tissue and picked up by the photodetector, preferably also positioned in the sensor, are converted into electronic signals (or photoplethysmographic signals or photoplethysmographic data) that are used to calculate the desired blood analyte levels and/or hemodynamic parameters. These values are then output for use by a clinician or other end user.

A device which generates light to be emitted into the tissue and detects and processes the photoplethysmographic signals (or data) emitted by the tissue, to measure the levels of various blood analytes and/or various hemodynamic parameters, is a photoplethysmographic device. A photoplethysmographic device includes a photoplethysmographic monitor combined with a photoplethysmographic sensor. The first widely used commercial photoplethysmographic device was a pulse oximeter, a photoplethysmographic device designed to noninvasively measure, at least, arterial blood oxygen saturation. This device is now used in almost all areas of medicine.

Preferably, the sensor is connected to the monitor by a patient cable that has a proximal connector to allow it to be removably connected to the monitor. Additionally, the patient cable may have a connector on its distal end to connect to, and disconnect from, the sensors.

Laser-based pulse oximeters, photoplethysmographic devices that use at least one laser light source to generate the light used to probe the tissue, provide some distinct advantages over conventional pulse oximeters that only use light emitting diodes (or LEDs) to generate the light used to probe the tissue in the sensor. Laser light is highly monochromatic, compared to LED light, providing greater measurement precision and allowing discrimination and measurement of blood analytes that may not be possible to measure with LED-based systems.

The use of lasers in photoplethysmography, however, also presents certain challenges. If lasers are to be used in a photoplethysmographic device, and if those lasers are to be positioned somewhere other than integral to the sensor (the photoplethysmographic sensor), then the light generated by the lasers must be communicated, or transmitted, to the sensor where it can exit the sensor and be incident on a tissue-under-test on which the sensor is positioned for monitoring.

If it is desirable to have a photoplethysmographic sensor that can be removably attached to the distal end of the patient cable, there are a number of constraints this places on the sensor and the sensor connector design. These constraints include delivering laser light, possibly in combination with LED light, to the tissue in the sensor; providing a connection system that allows the sensor to be connected and/or disconnected from the distal end of the patient cable; ensuring that the connection between the sensor and the patient cable distal connector is firm and secure for monitoring; and providing electrical interconnection between the electrical elements in the sensor and the patient cable. One additional constraint is that it would be better, in a clinical setting, if the connection between the sensor and the patient cable distal connector could be engaged, or disengaged, while the sensor is already on the tissue, without having to apply undo force on the tissue. It is the design of a sensor and cable interconnection that meets the above constraints that is the subject of this invention.

### SUMMARY OF THE INVENTION

The present invention overcomes the problems and disadvantages associated with current strategies and designs and provides new systems and methods of manufacturing photoplethysmographic sensor connectors.

A preferred embodiment of the invention is directed to a photoplethysmographic sensor for a laser-based pulse oximeter. The photoplethysmographic sensor has an enclosure and a sensor connector, the sensor connector including a stationary portion and a sliding portion, the stationary portion fixed in position with respect to the enclosure, the sliding portion coupled to the stationary portion and free to slide with respect to the stationary portion, the sensor connector stable in at least an open position and an engaged position and movable between the open and engaged positions through application of an external force, the sensor connector adapted to retain a distal connector when in the engaged position, the sensor connector adapted to move from the open position to the engaged position upon the stationary portion and the sliding portion being pinched together, wherein the stationary portion and the sliding portion are adapted to be pinched together without an application of pressure on the enclosure, and the sensor connector adapted to move from the engaged position to the open position upon the stationary portion and the sliding portion being pulled apart, wherein the pulling apart motion does not require pressure on the enclosure.

Preferably, the sensor connector provides tactile feedback when it is slid into the engaged position. In a preferred embodiment, the sensor connector provides auditory feedback when it is slid into the engaged position. Preferably, pinching the sensor connector into the engaged position actively forces the distal connector against the stationary portion and, once in the engaged position, maintains a force between the distal connector and the stationary portion. The force maintained between the distal connector and the stationary portion is preferably at least 1.766 newtons.

Preferably, the enclosure and sensor connector provide an opening to accept a light emitting portion of the distal connector. In a preferred embodiment, the enclosure and/or sensor connector provides a transparent window to mate with a light emitting portion of the distal connector. Preferably, the sensor connector includes at least two electrical contacts. The distal connector preferably includes at least two electrical contacts that mate with the at least two electrical contacts of the sensor connector.

Another embodiment of the invention is directed to a method of assembling a photoplethysmographic sensor for a laser-based pulse oximeter. The method includes the steps of coupling a stationary portion of a sensor connector on an enclosure such that the stationary portion does not move with respect to the enclosure, interlocking a movable portion of the sensor connector onto the stationary portion of the sensor connector such that the sliding portion is free to slide with respect to the stationary portion, wherein the interlocking connection of the stationary portion and the sliding portion provides at least a stable open position and a stable engaged position and the sensor connector is movable between the two positions through application of an external force, accepting insertion of a distal connector in the open position, retaining the distal connector in the engaged position, wherein the stationary portion and the sliding portion move from the open position to the engaged position with a pinching motion of the stationary portion and the sliding portion, wherein the pinching motion does not require pressure on the enclosure, and wherein the stationary portion and the sliding portion move from the engaged position to the open position by a pulling apart motion of the stationary portion and the sliding portion, wherein the pulling apart motion does not require pressure on the enclosure.

Preferably, the method also includes providing a tactile feedback when sliding the stationary portion and the sliding portion to the engaged position from the open position or when sliding the stationary portion and the sliding portion from the open position to the engaged position. In a preferred embodiment, the method further includes providing audible feedback when sliding the stationary portion and the sliding portion to the engaged position from the open position or when sliding the stationary portion and the sliding portion from the open position to the engaged position.

Preferably, the method further includes forcing the distal connector against the stationary portion when the sliding portion is moved from the open position to the engaged position and, once in the engaged position, maintaining a force between the retained distal connector and the stationary portion. The force maintained is preferably between the distal connector and the stationary portion is at least 1.766 newtons. Preferably the method further comprises accepting a light emitting portion of the distal connector in at least one of the enclosure and the sensor connector. Preferably, the method further includes positioning a transparent window within in at least one of the enclosure and the sensor connector to mate with a light emitting portion of the distal connector. In a preferred embodiment, the method further comprises positioning at least two electrical contacts in the sensor connector. Preferably, the method further comprises positioning at least two electrical contacts that mate with the at least two electrical contacts of the sensor connector in the distal connector.

Other embodiments and advantages of the invention are set forth in part in the description, which follows, and in part, may be obvious from this description, or may be learned from the practice of the invention.

### BRIEF DESCRIPTION OF THE DRAWINGS

The foregoing features of embodiments will be more readily understood by reference to the following detailed description, taken with reference to the accompanying drawings, which show:

FIG. 1: Photoplethysmographic sensor including enclosure and sensor connector with patient cable distal connector.

FIG. 2: Photoplethysmographic sensor including enclosure and sensor connector with patient cable distal connector, detailed view.

FIG. 3: Sensor (photoplethysmographic sensor) connector showing the sliding portion in the open position and (in dashed form) in the engaged positions.

FIG. 4a: Sensor connector in the open position with distal connector (patient cable distal connector) in position to be engaged, viewed from above.

FIG. 4b: Sensor connector with distal connector engaged, viewed from above.

FIG. 5a: Sensor connector in the open position with distal connector not yet engaged, viewed from below.

FIG. 5b: Sensor connector in the engaged position with distal connector engaged, viewed from below.

FIG. 6a: Alternate sensor connector configuration with sliding portion in the open position, viewed from below.

FIG. 6b: Alternate sensor connector configuration with sliding portion in the engaged position, viewed from below.

FIG. 7: Photoplethysmographic sensor including fingerclip style enclosure and sensor connector.

### DETAILED DESCRIPTION OF THE INVENTION

As embodied and broadly described herein, the disclosures herein provide detailed embodiments of the invention. However, the disclosed embodiments are merely exemplary of the invention that may be embodied in various and alternative forms. Therefore, there is no intent that specific structural and functional details should be limiting, but rather the intention is that they provide a basis for the claims and a representative basis for teaching one skilled in the art to variously employ the present invention.

Photoplethysmographic devices typically use LEDs to generate the light that is used to probe the tissue-under-test (tissue), but the use of LED light creates limitations on how accurate and precise the photoplethysmographic measurements can be due to the wide spectral bandwidth of the LED light sources. Additionally, inaccuracies have been found in LED-based pulse oximeters due to highly melanated skin, caused by the filtering effect of the LED light by the melanin in the skin. The broadband LED light passing through the melanin effectively shifts the spectral content of the light, causing a bias error in the oxygen saturation measurements.

To improve the accuracy and precision of photoplethysmographic, or pulse oximetry, measurements, a photoplethysmographic device can use one or more laser light sources to generate the light used to probe the tissue. Photoplethysmographic devices that use laser light to probe the tissue are laser-based pulse oximeters, or laser-based photoplethysmographic devices. Note that these laser-based devices may use multiple laser light sources, in combination with one or more LED light sources, to generate the light that is used to probe the tissue.

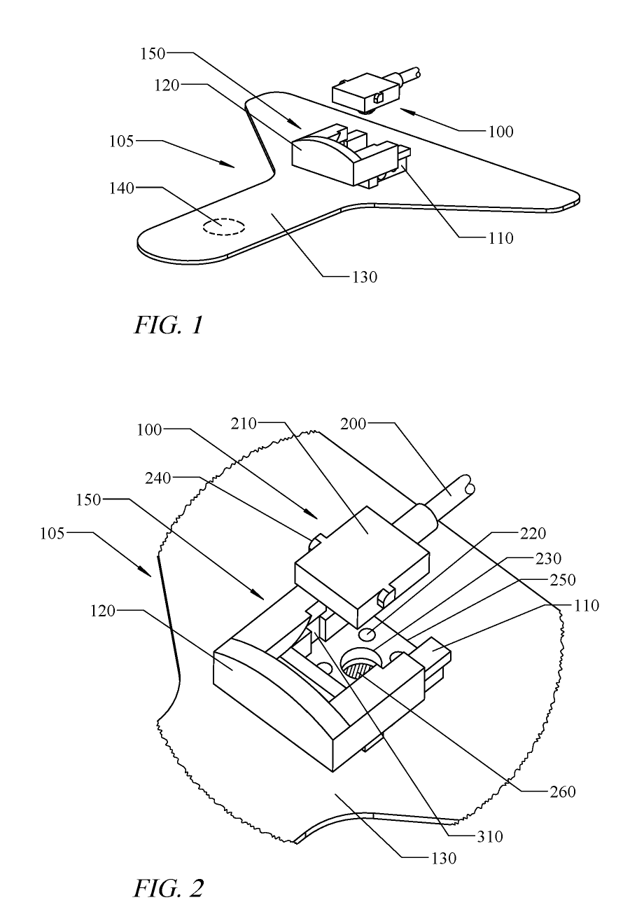

Because lasers, along with the electromechanical mounts that lasers require, can be physically too large to practically incorporate directly into a sensor (photoplethysmographic sensor), light from the lasers may be transmitted via one or more light guides, preferably optical fibers, to the sensor where that light can then be emitted by the sensor to be incident on the tissue that is placed in the sensor for making photoplethysmographic measurements. One embodiment of a tape-on photoplethysmographic sensor 105 is shown in FIG. 1.

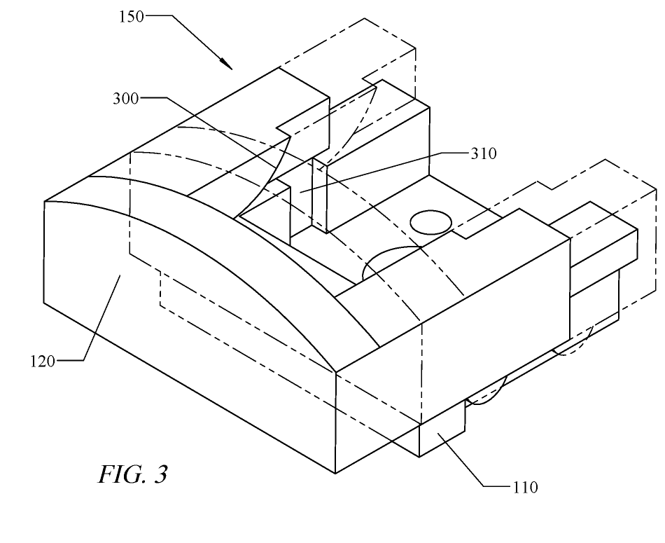

Sensor 105 includes tape-on enclosure 130. Enclosure 130 captures, or holds, the stationary portion 110 of sensor connector 150 and a photodetector 140. In the view shown in FIG. 1 the active area of the photodetector 140 would be visible on the bottom side (the side not shown) of enclosure 130. Enclosure 130 is designed to be wrapped around a finger or toe such that, when in position on the tissue for monitoring, opening 230 (FIG. 2) in sensor connector 150 and enclosure 130 is approximately across the tissue from the active area side of photodetector 140. While sensor 105 is preferably taped into position, other fixture methods can be used to attach sensor 105 to the patient, such as hook-and-loop connections or adhesive applied to the skin facing side of enclosure 130.

Photodetector 140 may, alternatively, be a light guide (such as an optical fiber) which conducts light from the sensor to a photodetector which can then be positioned at a distance from the sensor. This sensor configuration may be appropriate for sensors used in a magnetic resonance imaging environments where eliminating all electrical elements from the sensor may be necessary.

Sensor connector 150 includes stationary portion 110 and sliding portion 120. These two parts are preferably injection molded plastic parts; however, they can be made of another material and/or through another process. It is the stationary portion 110 that is captured, or fixed in position, by enclosure 130. Sliding portion 120 once hooked, or coupled, onto stationary portion 110 during manufacturing can be moved, or slid, with respect to stationary portion 110, to at least two stable positions, an open position and an engaged position. In FIG. 1 sensor connector is shown in the open position, ready for insertion of the distal connector 210 (FIG. 2) on the distal end of patient cable 100, shown above sensor connector 150 and oriented properly for insertion into sensor connector 150.

FIG. 2 is a detailed view of the tape-on sensor 105 and distal end of patient cable 100 from a slightly different viewing angle. In this view a thin printed circuit board 250, sits directly on top of the base of stationary portion 110 which is fixed in position with respect to enclosure 130. Printed circuit board 250 provides electrical contacts 220 for interconnection with electrical contacts in distal end of patient cable 100. Printed circuit board 250 is preferably in data communication with photodetector 140. The distal end of patient cable 100 includes the distal connector (patient cable distal connector) 210, and the end of cable 200 that can include electrical and optical signal conductors, electrical shielding, strengthening members, and an overall protective jacket. The other end of patient cable 100 preferably has a proximal connector for connection to the photoplethysmographic monitor. Preferably there are at least two pairs of electrical contacts in distal connector 210 that engage the electrical contacts 220 of sensor connector 150 (with corresponding conductors inside cable 200) for communication of the photoplethysmographic signals, received by the photodetector, back to the monitor. Other electrical connections may be required for communicating with a memory element, calibration element, or other electronics in sensor 105.

Wings 240, one on each side of distal connector 210, slide into slots 310 (only one slot visible in this view) in stationary portion 110 of sensor connector 150. Once distal connector 210 is placed into stationary portion 110, sliding portion 120 is moved from the open position, as shown in FIG. 2, to an engaged position that locks distal connector 210 into sensor connector 150.

Opening 230 may have a transparent window 260 at the bottom of the opening in enclosure 130 or stationary portion 110 to act as a sterile or sanitary barrier to microbes on distal connector 210. Window 260 abuts, or mates, with the end of the light emitting portion of distal connector 210 when distal connector 210 is held in sensor connector 150 and sliding portion 120 is in the engaged position.

FIG. 3 shows the two parts of sensor connector 150, the stationary portion 110 and the sliding portion 120. Sliding portion 120, is shown in the open position, using solid lines, and in the engaged position using dashed lines. Once distal connector 210 is set into sensor connector 150 such that wings 240 are positioned in slots 310, moving the sliding portion 120 from the open position to the engaged position causes the sloped edges 300 to drive, or force, distal connector 210 down against the base of stationary portion 110 ensuring good electrical connection between electrical contacts 220 on the sensor connector 150 and matching electrical contacts on distal connector 210.

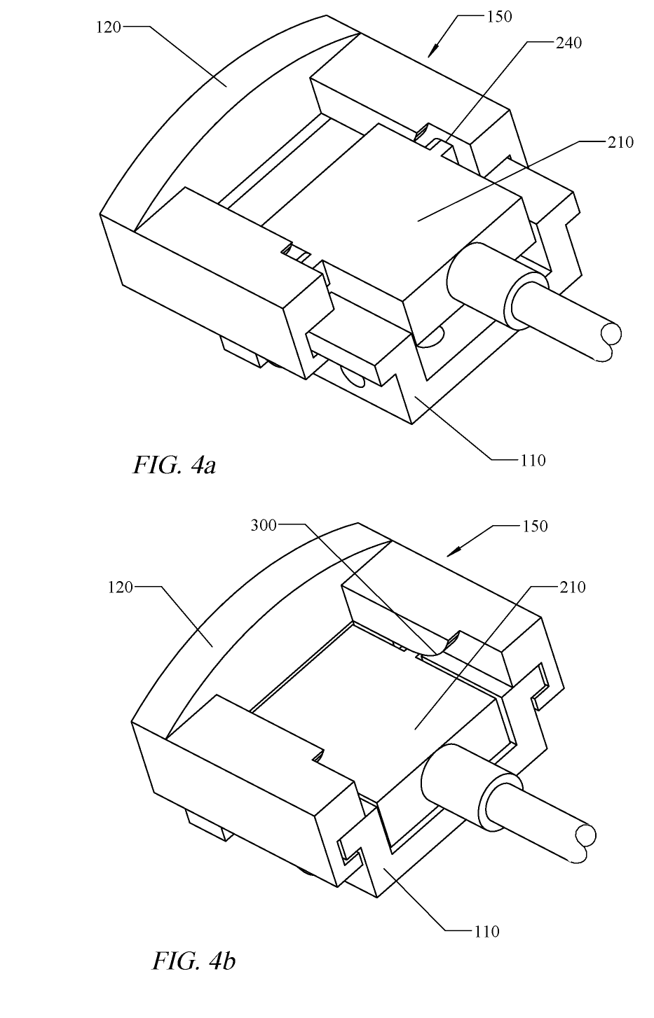

FIG. 4a shows sensor connector 150 with sliding portion 120 in the open position and with distal connector 210 resting on stationary portion 110 of sensor connector 150. Wings 240 are slightly engaged at the top of the slots 310 in stationary portion 110. FIG. 4b shows sensor connector 150 with sliding portion 120 in the engaged position, with distal connector 210 pressed down against the stationary portion 110. To move sliding portion 120 from the open position to the engaged position, the user of the sensor can simply apply an external force to squeeze, or pinch, the sliding portion 120 and the stationary portion 110 together without needing to apply downward pressure on sensor connector 150. In sliding these two portions of sensor connector 150 together, ramp 300 drives distal connector 210 down into and against stationary portion 110. This eliminates the need to put pressure on the tissue in sensor 105 if the sensor is already on a patient when distal connector 210 is connected. Similarly, pulling the sliding portion 120 away from the stationary portion 110 to move from the engaged position to the open position does not require pressing down on, or pulling up on, sensor connector 150.

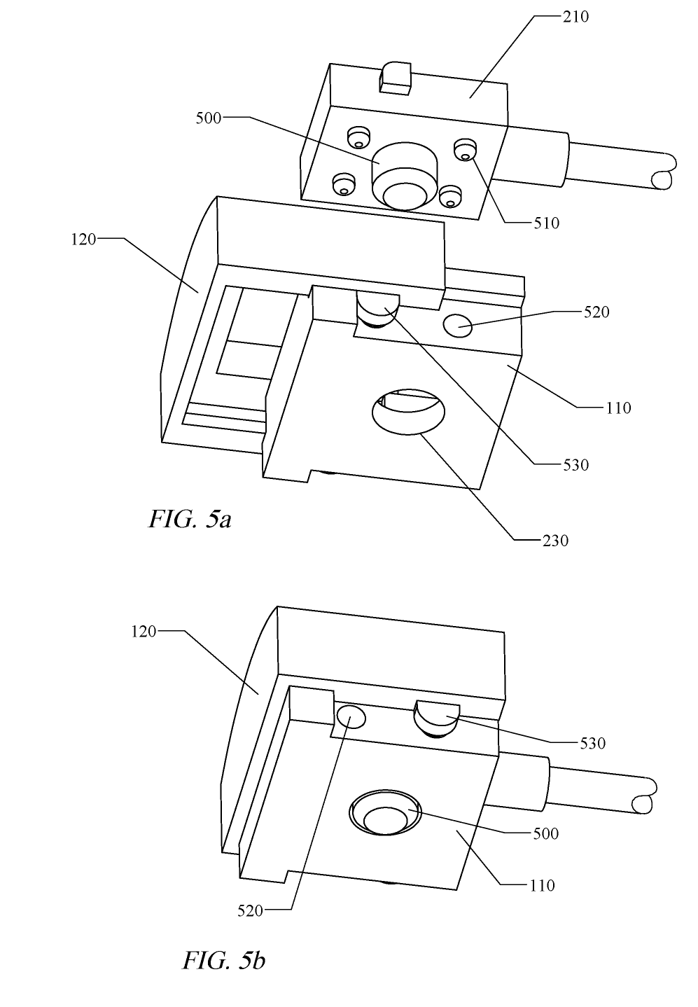

In FIG. 5a electrical contacts 510 are visible on the underside of distal connector 210, and sliding portion 120 is in the open position. In a preferred embodiment, electrical contacts 510 are spring-loaded pins that recess back into distal connector 210 when pressed against the contacts on circuit board 250 in stationary portion 110. In the open position, as shown in FIG. 5a, bumpouts 530 are positioned in the “open position detents” of detents 520. (There is a second bumpout and set of two detents on the other side of stationary portion 110 not visible in this figure.) Light pipe 500, the light emitting portion of distal connector 210, protrudes from the underside of distal connector 210 and, when distal connector 210 is fixed in sensor connector 150, light pipe 500 will be positioned in hole 230 in stationary portion 110 as shown in FIG. 5b. To maintain good electrical contact between electrical contacts 510 and electrical contacts 220, sensor connector 150 is capable of applying a minimum of 1.766 newtons of force on the distal connector 210 toward stationary portion 110. This force may be created by opposing the force of the spring-loaded pins if employed by the electrical contacts 510, by a spring compressed between distal connector 210 and sliding portion 120 when in the engaged position, or by a force created by an interference fit between distal connector 210 and sliding portion 120 when in the engaged position.

Light pipe 500 is needed to deliver light, generated at a distance from the sensor, to a tissue-under-test positioned in sensor 105. Preferably, laser light from one or more lasers is transmitted by light guides, typically fiber optics inside cable 200 of patient cable 100, to distal connector 210 and out through light pipe 500. Light emitted by light pipe 500 includes laser light from one or more laser light sources and may include LED light from one or more LED light sources.

FIG. 5b shows distal connector 210 fully inserted into sensor connector 150 with sliding portion 120 in the engaged position. In this position bumpouts 530 are in the “engaged position detents” of detents 520. The bumpouts 530 and detents 520 hold the sliding portion 120 stable when it is in either the open position or the engaged position.

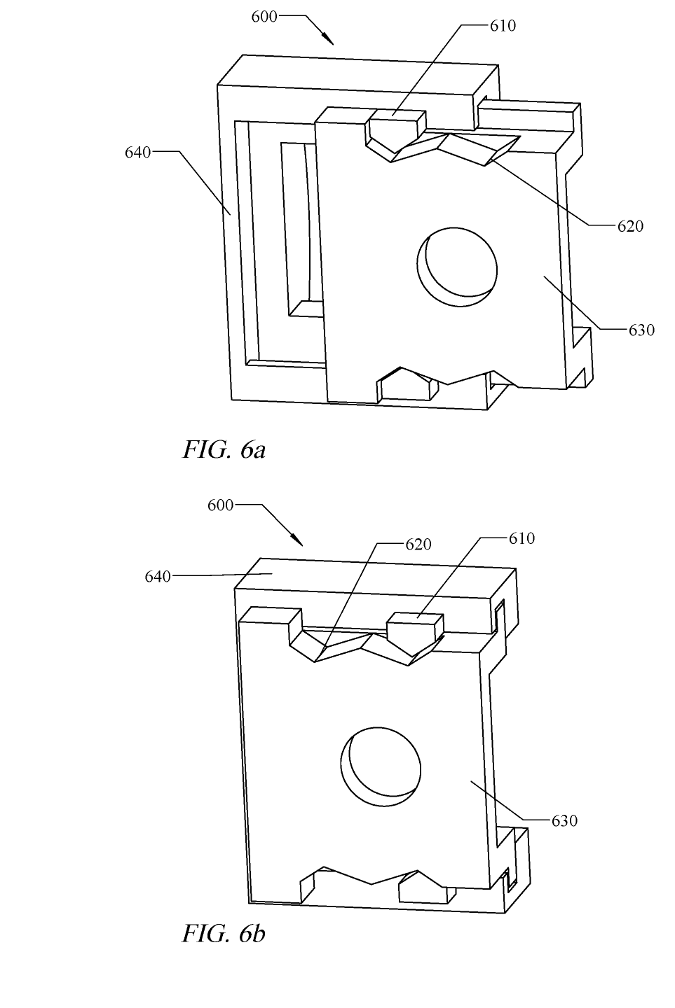

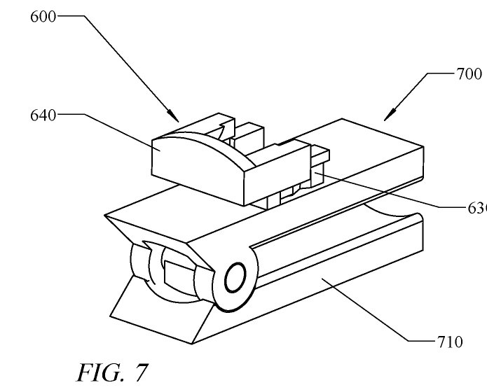

An alternate mechanical structure for the sensor connector is shown in FIG. 6a and 6b. FIG. 6a shows a bottom view (looking from enclosure 130 upward) of sensor connector 600. In this embodiment the stationary portion 630 has two detents 620 on each side. Sliding portion 640 has bumpouts 610 on both of its sides. In FIG. 6a, sliding portion 640 is shown in the open position with bumpouts 610 in the “open position (left-hand in the figure) detents” of detents 620 of stationary portion 630.

The bottom view of sensor connector 600 shown in FIG. 6b shows sliding portion 640 in the engaged position with bumpouts 610 in the “engaged position (right-hand in the figure) detents” of detents 620. Because of the shape of detents 620 the sliding portion is stable in either the open or the engaged positions. Sliding portion 640 cannot travel further than the open position due to interference between the side of bumpout 610 on sliding portion 640 and the wall on stationary portion 630 that abuts bumpout 610. In the engaged position shown in FIG. 6b the sliding portion 640 cannot slide further to the right (in the figure) due to interference between the left-hand (in the figure) wall of sliding portion 640 and the left-hand (in the figure) wall of stationary portion 630.

Sensor connector embodiments 150 or 600, or other similar modifications thereof, can be used with any number of different enclosure styles. FIG. 7 shows a fingerclip style sensor 700 including sensor connector 600. In this configuration fingerclip 710 is the enclosure that is fixedly attached to stationary portion 630 with sliding portion 640 shown in the open position.

Regardless of the exact configuration of the sensor connector and enclosure, preferably the distal connector 210 is drawn down into the sensor connector 150 (or equivalently 600) through a sliding motion of sliding portion 120 (or equivalently 640) with respect to stationary portion 110 (or equivalently 630) wherein the sliding motion requires no downward pressure, on the enclosure. Thus, if senor 105 (or equivalently 700) is already on a patient’s tissue, no pressure need be applied to the tissue to engage or open the sensor connector 150 (or equivalently 600).

Additionally, tactile and auditory feedback to the user is created by feeling the sliding portion 120 (or equivalently 640) snap into place against stationary portion 110 (or equivalently 630) as the bumpouts 530 (or 610) engage in the detents 520 (or 620) and/or from the two portions of the sensor connector 150 (or 600) reaching the end of the constrained travel between the two positions (open to engaged or engaged to open) where plastic slaps against plastic (assuming that the parts are made of plastic, but the same would hold true if an alternate material was used).

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims. Furthermore, the term “comprising of” includes the terms “consisting of” and “consisting essentially of.”

1. A photoplethysmographic sensor for a laser-based pulse oximeter, comprising:
an enclosure and a sensor connector;
the sensor connector including a stationary portion and a sliding portion;
the stationary portion fixed in position with respect to the enclosure;
the sliding portion coupled to the stationary portion and free to slide with respect to the stationary portion;
the sensor connector stable in at least an open position and an engaged position and movable between the open and engaged positions through application of an external force;
the sensor connector adapted to retain a distal connector when in the engaged position;
the sensor connector adapted to move from the open position to the engaged position upon the stationary portion and the sliding portion being pinched together, wherein the stationary portion and the sliding portion are adapted to be pinched together without an application of pressure on the enclosure; and
the sensor connector adapted to move from the engaged position to the open position upon the stationary portion and the sliding portion being pulled apart, wherein the pulling apart motion does not require pressure on the enclosure.

2. The device of claim 1, wherein the sensor connector provides tactile feedback when it is slid into the engaged position.

3. The device of claim 1 wherein the sensor connector provides auditory feedback when it is slid into the engaged position.

4. The device of claim 1, wherein pinching the sensor connector into the engaged position actively forces the distal connector against the stationary portion and, once in the engaged position, maintains a force between the distal connector and the stationary portion.

5. The device of claim 4, wherein the force maintained between the distal connector and the stationary portion is at least 1.766 newtons.

6. The device of claim 1, wherein the enclosure and sensor connector provide an opening to accept a light emitting portion of the distal connector.

7. The device of claim 1, wherein the enclosure and/or sensor connector provides a transparent window to mate with a light emitting portion of the distal connector.

8. The device of claim 1, wherein the sensor connector includes at least two electrical contacts.

9. The device of claim 8, wherein the distal connector includes at least two electrical contacts that mate with the at least two electrical contacts of the sensor connector.

10. A method of assembling a photoplethysmographic sensor for a laser-based pulse oximeter, comprising the steps of:
coupling a stationary portion of a sensor connector on an enclosure such that the stationary portion does not move with respect to the enclosure;
interlocking a movable portion of the sensor connector onto the stationary portion of the sensor connector such that the sliding portion is free to slide with respect to the stationary portion;
wherein the interlocking connection of the stationary portion and the sliding portion provides at least a stable open position and a stable engaged position and the sensor connector is movable between the two positions through application of an external force;
accepting insertion of a distal connector in the open position; and
retaining the distal connector in the engaged position;
wherein the stationary portion and the sliding portion move from the open position to the engaged position with a pinching motion of the stationary portion and the sliding portion, wherein the pinching motion does not require pressure on the enclosure; and
wherein the stationary portion and the sliding portion move from the engaged position to the open position by a pulling apart motion of the stationary portion and the sliding portion, wherein the pulling apart motion does not require pressure on the enclosure.

11. The method of claim 10, further comprising providing a tactile feedback when sliding the stationary portion and the sliding portion to the engaged position from the open position or when sliding the stationary portion and the sliding portion from the open position to the engaged position.

12. The method of claim 10, further comprising providing audible feedback when sliding the stationary portion and the sliding portion to the engaged position from the open position or when sliding the stationary portion and the sliding portion from the open position to the engaged position.

13. The method of claim 10, further comprising forcing the distal connector against the stationary portion when the sliding portion is moved from the open position to the engaged position and, once in the engaged position, maintaining a force between the retained distal connector and the stationary portion.

14. The method of claim 13, wherein the force maintained between the distal connector and the stationary portion is at least 1.766 newtons.

15. The method of claim 10, further comprising accepting a light emitting portion of the distal connector in at least one of the enclosure and the sensor connector.

16. The method of claim 10, further comprising positioning a transparent window within in at least one of the enclosure and the sensor connector to mate with a light emitting portion of the distal connector.

17. The method of claim 10, further comprising positioning at least two electrical contacts in the sensor connector.

18. The method of claim 17, further comprising positioning at least two electrical contacts that mate with the at least two electrical contacts of the sensor connector in the distal connector.
